(19) United States
(12) Patent Application Publication
(10) Pub. No.: US 2021/0178015 A1
(43) Pub. Date: Jun. 17, 2021

(54) METHOD TO PRODUCE A PROSTHETIC COMPONENT, AND PROSTHETIC COMPONENT THUS PRODUCED

(71) Applicant: LIMACORPORATE S.P.A., San Daniele Del Friuli (IT)

(72) Inventor: Michele PRESSACCO, Martignacco (IT)

(21) Appl. No.: 16/973,816

(22) PCT Filed: Jun. 20, 2019
(86) PCT No.: PCT/IT2019/050147

§ 371 Date: Dec. 10, 2020

(30) Foreign Application Priority Data
Jun. 20, 2018 (IT) 102018000006531

(51) Int. Cl. A61L 27/04; A61L 27/06; A61L 27/30

(52) U.S. Cl. CPC A61L 27/045; A61L 27/06; A61L 27/306; A61L 2400/18; A61L 2420/08; A61L 2430/02; A61L 2430/24; A61L 2420/02

(57) ABSTRACT

Method to produce a single-piece prosthetic component (110, 210, 310, 410, 510, 610, 620, 710, 810, 910) that comprises making available a substrate (12) and making a coating layer (14) thereon.

### FIELD OF THE INVENTION

Embodiments described here concern a method to produce a prosthetic component and the corresponding prosthetic component thus produced, for example usable to restore animal and more specifically human joints. For example, embodiments described here can find application in the medical sector, in particular orthopedic, for prosthetic implants and possibly also prostheses that act as bone substitutes.

### BACKGROUND OF THE INVENTION

In the medical sector, in particular to produce prosthetic implants, it is known to use cobalt-based metal alloys, in particular cobalt-chromium, such as the CoCrMo alloy, as well as titanium-based metal alloys, such as Ti6Al4V (abbreviated Ti64).

It is known that cobalt-based metal alloys have properties of hardness, wear resistance and adequate tribological features (with reference to standard ASTM F2083), and that, therefore, they are used in the bio-medical, in particular orthopedic, field to produce prosthetic components or implants subject to cyclical loads of wear and friction. Typically, cobalt-based metal alloys are used for cemented prostheses, whereas they are not usually used for prostheses for uncemented use, since they can typically denote poor osseointegration capacity, especially if compared with other materials, such as the titanium-based metal alloys as above.

It is known, in fact, that the titanium-based metal alloys denote high biocompatibility and high capacity to firmly attach to the bone, that is osseointegration, and are, therefore, used to produce prostheses for uncemented use, that is with direct contact with the bone matrix, that is prostheses that are firmly attached to the bone, for example being configured with a porous or trabecular structure. See for example EP-B-2.164.428 in the name of the Applicant. These titanium-based metal alloys, however, could denote poor abrasion resistance properties and poor tribological features (ASTM F2083) and be unsuitable to produce prosthetic components subject to cyclical loads of wear and friction or for connection with the joints.

However, attempts have been made in the state of the art to use the two types of alloys, cobalt-based and titanium-based respectively, in a prosthesis, in order to exploit their different properties. A first approach consists in producing modular prostheses, in which the prosthesis is formed by the coupling of two separate prosthetic modules, made in the different metal materials in question: elements of the prosthesis made with the two different alloys are directly associated with each other, or by means of one or more intermediate connection elements or adapters. This first approach has first of all the disadvantage of the onset of “fretting”, or friction, phenomena at the interface in direct contact between the two metal surfaces with the risk of releasing metal particles and metal ions at the interface between the two materials, which can propagate in the tissues of the organism in which the prosthesis is implanted, in fact increasing the concentration of metals in the blood, with the consequent risks for the health of the patient. Furthermore, the modular prostheses, because of how they are produced, can be more voluminous and bulkier compared to a traditional prosthesis and, therefore, require the removal of a large quantity of bone tissue in order to be implanted. Furthermore, in this context, it should be noted that, in particular joint areas, such as for example in the femoral component of the knee, there may not be sufficient space to make a modular prosthesis, without removing too much bone tissue. For this reason, it would be desirable for the prosthesis to have a reduced bulk and to allow to reduce the quantity of bone tissue to be removed, especially in these specific operating situations.

A second possible approach consists in making a single-piece, or monolithic, prostheses, which provides to constrain in a definitive manner the two different metal materials. This second approach has the advantage, compared to the modular prostheses as above, of avoiding “fretting” phenomena and making available less voluminous and bulky prostheses. Normally, this second approach provides to achieve the constraint by fusion of the two different alloys. However, it is known that, for reasons related to the chemical-physical properties, above all the different melting temperatures, and mechanical properties, and to their different mechanical behaviour, it is difficult to couple the cobalt-based alloys and the titanium-based alloys in question with each other, in order to make a suitable prosthetic component. This is particularly true if it is provided to melt the two alloys, since the titanium-based alloy is more reactive. Consequently, even different elements of a prosthetic component, made respectively with one and the other alloy, are difficult to directly couple with each other in a prosthesis that is stable, functional and safe for the patient. In particular, a prosthetic component made in this manner would not guarantee the stability and functionality of the prosthesis itself, possibly presenting the formation of cracks at the interface between the two metal alloys, which would make the prosthetic component made in this manner fragile and would, in fact, present serious risks for the health of a patient using this prosthetic component.

There is therefore the need to provide a method to produce a prosthetic component, and a corresponding prosthetic component, which overcome at least one of the disadvantages of the state of the art.

In particular, one purpose of the present invention is to provide a method to produce a prosthetic component, and a corresponding prosthetic component, which can combine, in a single-piece or single-component prosthetic component, usable as a prosthesis, or as a module of a modular prosthesis, cobalt-based metal alloys, in particular cobalt-chromium, and titanium-based alloys, so as to exploit both the osseointegration and biocompatibility properties of titanium-based metal alloys, and also at least the tribological features, in terms of hardness and wear resistance of cobalt-based metal alloys, in particular cobalt-chromium, and possibly also the mechanical features, without compromising the stability, resistance and functionality of the prosthesis itself.

Another purpose of the present invention is to provide a method to produce a prosthetic component, and a corresponding prosthetic component, in which the titanium-based metal alloy is present at the bone interface, so as to exploit its biocompatibility and osseointegration properties, and the cobalt-based metal alloy, in particular cobalt-chromium, is present on the articular surfaces and/or in the core of the articular structure, so as to exploit its tribological properties, mechanical properties of hardness and strength.

Furthermore, the present invention aims to provide a prosthetic component produced with the above method which has high biocompatibility characteristics and which is at the same time mechanically resistant.

A further purpose of the present invention is to provide a single-piece prosthetic component which has a reduced bulk, allowing to reduce the quantity of bone tissue to be removed, thus being able to also be implanted in articular areas where it is not, in fact, possible to implant a modular prosthesis.

The Applicant has devised, tested and embodied the present invention to overcome the shortcomings of the state of the art and to obtain these and other purposes and advantages.

### SUMMARY OF THE INVENTION

The present invention is set forth and characterized in the independent claims. The dependent claims describe other characteristics of the invention, or variants to the main inventive idea.

In accordance with the above purposes, a method to produce a single-piece prosthetic component according to embodiments described here comprises:
making available a substrate made of a first titanium-based metal alloy;
making, on the substrate, a coating layer of a second cobalt-based metal alloy, in particular cobalt-chromium, by means of a Directed Energy Deposition (DED) process, or laser cladding.

Here and in the following description and claims, the expression “making a coating layer on the substrate” can be understood, depending on the embodiments, as both a method in which the coating layer is made directly on the substrate, in direct contact therewith, and also a method in which the coating layer is made above the substrate, without a 100% direct contact between the two different alloys, for example providing one or more intermediate layers between the substrate and the coating layer in which there is a progressive variation of the composition of the two alloys, from 100% to 0% and from 0% to 100% in a corresponding manner in the two directions, that is from the cobalt-based alloy to the titanium-based one, and vice versa.

From experimental tests the Applicant has discovered that making the coating layer by means of the DED process, or laser cladding, advantageously allows to make the coating layer on the substrate without the formation of cracks at the interface between the two alloys.

In this way, advantageously, the present invention allows to produce a prosthetic component which has both the high osseointegration properties of the first titanium-based metal alloy, and also the high structural properties and tribological features of the second cobalt-based metal alloy, in particular cobalt-chromium.

Further embodiments, combinable with all the embodiments described here, concern a method to produce a single-piece prosthetic component. In one embodiment the above method comprises:
making available a substrate made of a first cobalt-based metal alloy;
making, on the substrate, a coating layer of a second titanium-based metal alloy, by means of a Directed Energy Deposition (DED) process, or laser cladding.

According to possible embodiments, combinable with all the embodiments described here, the method in accordance with the present description provides to make, in physical and structural continuity between the substrate and the coating layer, one or more intermediate layers made with a material having a functional gradient, with a mixed composition of a cobalt-based and titanium-based alloy, by means of a Directed Energy Deposition (DED) process, or laser cladding.

Embodiments of the present invention also concern a single-piece prosthetic component obtainable by means of embodiments of the method in accordance with the present description.

Here and in the present description, a single-piece prosthetic component is intended as a prosthetic component in a single-piece, which can be used as a single-piece or single-component prosthesis in itself, or as a module of a modular prosthesis.

Embodiments of the present invention, also, concern a single-piece prosthetic component comprising a substrate made of a first titanium-based metal alloy and, on said substrate, a coating layer made of a second cobalt-based metal alloy, in particular cobalt-chromium, in which the coating layer is obtained by means of a Directed Energy Deposition (DED) process, or laser cladding.

Further embodiments concern a method to produce a single-piece prosthetic component. In one embodiment, the above method comprising:
making available a substrate, or core, made of a first cobalt-based metal alloy;
making, on the substrate, a coating layer of a second titanium-based metal alloy, by means of a Directed Energy Deposition (DED) process, or laser cladding.

Further embodiments concern a single-piece prosthetic component comprising an internal substrate, or core, made of a first cobalt-based metal alloy and, on the substrate, a coating layer of a second titanium-based metal alloy, which completely covers the internal substrate, or core, the coating layer being obtained by means of a Directed Energy Deposition (DED) process, or laser cladding.

These and other aspects, characteristics and advantages of the present disclosure will be better understood with reference to the following description, drawings and attached claims. The drawings, which are integrated and form part of the present description, show some embodiments of the present invention and, together with the description, are intended to describe the principles of the disclosure.

The various aspects and characteristics described in the present description can be applied individually where possible. These individual aspects, for example aspects and characteristics present in the description or in the attached dependent claims, can be the object of divisional applications.

It is understood that any aspect or characteristic that is discovered, during the patenting process, to be already known, shall not be claimed and shall be the object of a disclaimer.

### BRIEF DESCRIPTION OF THE DRAWINGS

These and other characteristics of the present invention will become apparent from the following description of some embodiments, given as a non-restrictive example, with reference to the attached drawings wherein:

FIG. 1 is a schematic section view of a first embodiment of a prosthetic component according to the present invention;

FIG. 2 is a schematic section view of a possible variant of FIG. 1;

FIG. 3 is a schematic section view of a further variant of FIG. 1;

FIG. 4 is a schematic section view of a second embodiment of a prosthetic component according to the present invention;

FIG. 5 is a schematic section view of a third embodiment of a prosthetic component according to the present invention;

FIGS. 6 and 7 are schematic, section and perspective views respectively, of a fourth embodiment of a prosthetic component according to the present invention;

FIGS. 8 and 9 are schematic views of a fifth embodiment of a prosthetic component according to the present invention;

FIG. 10 is a schematic section view of a sixth embodiment of a prosthetic component according to the present invention;

FIG. 11 is a schematic section view of a seventh embodiment of a prosthetic component according to the present invention;

FIG. 12 is a schematic section view of an eighth embodiment of a prosthetic component according to the present invention;

FIGS. 13 and 14 are schematic, section and perspective views respectively, of a ninth embodiment of a prosthetic component according to the present invention;

FIGS. 15 and 16 are schematic, section and perspective views respectively, of a tenth embodiment of a prosthetic component according to the present invention;

FIG. 17 is a schematic view of an eleventh embodiment of a prosthetic component according to the present invention;

FIG. 18 is a schematic section view along the line XVIII-XVIII of the prosthetic component of FIG. 17;

FIG. 19 is a schematic partial section view of the prosthetic component of FIG. 2 on a support;

FIG. 20 is a schematic perspective view of FIG. 19.

To facilitate comprehension, the same reference numbers have been used, where possible, to identify identical common elements in the drawings. It is understood that elements and characteristics of one embodiment can conveniently be incorporated into other embodiments without further clarifications.

### DETAILED DESCRIPTION OF SOME EMBODIMENTS

We will now refer in detail to the various embodiments of the present invention, of which one or more examples are shown in the attached drawing. Each example is supplied by way of illustration of the invention and shall not be understood as a limitation thereof. For example, the characteristics shown or described insomuch as they are part of one embodiment can be adopted on, or in association with, other embodiments to produce another embodiment. It is understood that the present invention shall include all such modifications and variants.

Before describing these embodiments, we must, also, clarify that the present description is not limited in its application to details of the construction and disposition of the components as described in the following description using the attached drawings. The present description can provide other embodiments and can be obtained or executed in various other ways. We must, also, clarify that the phraseology and terminology used here is for the purposes of description only, and cannot be considered as limitative.

Embodiments described here, using the attached drawings, concern a method to produce a single-piece prosthetic component, indicated with reference 110, 210, 310, 410, 510, 610, 620, 710, 810, 910, 1010 in the various embodiments in accordance with the present description. The single-piece prosthetic component can be used as a single-piece prosthesis in itself, or as a module of a modular prosthesis.

In general, embodiments described here provide to make available a single-piece prosthetic component in which a variation of material is provided, from a titanium-based metal alloy to a cobalt-based metal alloy, in which the titanium-based metal alloy is provided at the bone interface. In particular, the titanium-based metal alloy can be provided at the bone interface, and the cobalt-based metal alloy can be provided as a material of the articular surface. Alternatively, the titanium-based metal alloy can be provided at the bone interface, and the cobalt-based metal alloy can be provided as an internal material that constitutes a core with a hard structure.

The titanium-based metal alloy can possibly be configured to promote osseointegration, for example it can be of the porous or trabecular type, for example a porous or trabecular metal structure obtained by means of an additive manufacturing technique, and/or provided with a coating, for example coated with plasma spray technique.

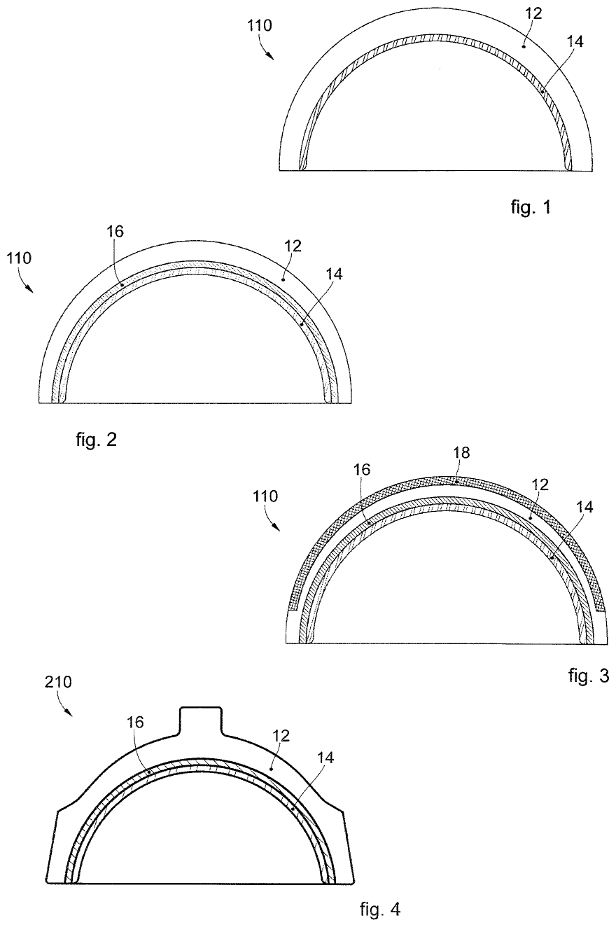

According to some embodiments, described using FIGS. 1-16, the method comprises at least:
making available a substrate 12 made of a first titanium-based metal alloy;
making, on said substrate 12, a coating layer 14 in a second cobalt-based metal alloy, in particular cobalt-chromium, by means of a Directed Energy Deposition (DED) process, or laser cladding.

According to the present description, the step of making the coating layer 14 is performed, as we said, by means of a Directed Energy Deposition, or DED, process, or laser cladding. Here and in the present description, with the expression “Directed Energy Deposition”, DED, we mean both a process using a laser beam (Light Amplification by Stimulated Emission of Radiation), therefore a process belonging to the family of “Laser Cladding” type processes, and also a process performed by means of an electron beam, for example by means of EBAM® technology available from Sciaky, Inc. In particular, by means of the Directed Energy Deposition, or DED, process the coating layer 14 is made by depositing the second cobalt-based metal alloy in powder form on the substrate 12 and its simultaneous melting, for example by means of a laser beam or electron beam striking on the powder.

According to possible embodiments, combinable with all the embodiments described here, the above substrate 12 is able to act as a bone interface, being made of a material configured to promote osseointegration.

For example, the substrate 12 can advantageously be made with a porous or trabecular structure, as described for example in EP-B-2.164.428 in the name of the Applicant.

In particular, the first titanium-based metal alloy can be Ti6Al4V, also called Ti64.

According to some embodiments, the method further provides to make the coating layer 14, on the substrate 12, in a second cobalt-based metal alloy, in particular cobalt-chromium, which for example can be CoCrMo.

In this way, advantageously, it is possible to produce a prosthetic component which has both the high osseointegration properties of the first titanium-based metal alloy, and also the high structural properties and tribological features of the second cobalt-based metal alloy, in particular cobalt-chrome.

According to the present description, the substrate 12 can be heated to the temperature necessary to avoid the formation of cracks in the substrate 12 and in the coating layer 14 during the production process. According to possible embodiments, the substrate 12 can be heated to the necessary temperature by means of the same laser beam or electron beam used for the DED process, or alternatively by independent heating means.

In some embodiments, heating, for example by means of the same DED process, or obtained in an autonomous manner, therefore allows to avoid the formation of cracks at the interface between substrate 12 and coating layer 14, allowing the production of single-piece prosthetic components without the need to use a third component as a binder.

According to possible embodiments, the coating layer 14 can have a thickness comprised between 0.1 mm and 3 mm.

According to possible embodiments, the single-piece prosthetic component according to the present description comprises the substrate 12 made of a first titanium-based metal alloy and, on said substrate 12, the coating layer 14 of a second cobalt-based metal alloy, in particular cobalt-chromium. The coating layer 14, as we said, is advantageously obtained by means of a Directed Energy Deposition, DED, process or laser cladding.

According to possible embodiments, combinable with all the embodiments described here, the substrate 12 is obtained by means of a process of additive manufacturing and/or by conventional techniques, in particular sintering or metallurgical techniques of a subtractive type, such as molding, forging or suchlike.

Here and in the following description and claims, with the term “additive manufacturing” we mean techniques and technologies in which the finished product is formed without the need to melt the material in molds or to remove it from a raw mold.

For example, additive manufacturing can include any one of the following techniques: EBM (Electron Beam Melting), or electron beam fusion, SLM (Selective Laser Melting), DMSLS (Direct Metal Selective Laser Sintering), or selective laser sintering, or suchlike, in which the material in the form of powder, threads or granules is locally melted to produce the finished product. In these techniques, successive flat layers of determinate and limited thickness of powders of metal material are deposited and solidified. These techniques allow to achieve high levels of precision and the creation of desired structures.

The sequence of the layers above gradually makes the corresponding three-dimensional theoretical model of the substrate 12 and, generated by means of computer design tools, at the same time creates the desired porous, or trabecular, structure, that is, the desired cellular lattice. These techniques can be used to make the substrate 12. A specific, though non-limiting, implementation can provide to use the EBM technique to make the substrate 12. In a possible embodiment, the structure of the cellular lattice which obtains the porous, or trabecular, structure of the substrate 12, can have open cells, intercommunicating with each other, advantageously promoting osseointegration.

Alternatively or additionally, the substrate 12 can be made by conventional processes, for example sintering, or conventional metallurgical techniques of the subtractive type, such as molding, forging or suchlike.

According to possible embodiments, combinable with all the embodiments described here, the coating layer 14 is able to act as an articular structure, both directly toward the bone, and also toward other materials of artificial components of the prosthesis, according to the conformation of the prosthesis provided, being made of material configured to resist cyclical loads of wear and friction.

According to possible embodiments, combinable with all the embodiments described here, the above second cobalt-based metal alloy, in particular cobalt-chromium, is CoCrMo.

According to possible embodiments, described using for example FIG. 1, the above single-piece prosthetic component consists exclusively of the substrate 12 and of the coating layer 14.

According to further possible embodiments described using FIGS. 2-6 and combinable with all the embodiments described here, the above single-piece prosthetic component can comprise, in physical and structural continuity between the substrate 12 and the coating layer 14, one or more intermediate layers 16 made with a material having a functional gradient, with a mixed material, that is with a mixed composition of a cobalt-based and titanium-based alloy, obtained by means of a Directed Energy Deposition, DED, process, or laser cladding.

According to possible implementations, the one or more intermediate layers 16 each have a composition of Ti6Al4V—CoCrMo, according to the formula CoCrMo X%+Ti6Al4V (100−X)% in which X<100.

According to possible embodiments, combinable with all the embodiments described here, the above one or more intermediate layers 16 comprise:
a first intermediate layer 16 on the substrate 12, the first intermediate layer 16 having a composition of 12.5% CoCrMo and 87.5% Ti6Al4V:
a second intermediate layer 16 on the first intermediate layer 16, the second intermediate layer 16 having a composition of 25% CoCrMo and 75% Ti6Al4V;
a third intermediate layer 16 on the second intermediate layer 16, the third intermediate layer 16 having a composition of 37.5% CoCrMo and 62.5% Ti6Al4V;
a fourth intermediate layer 16 on the third intermediate layer 16, the fourth intermediate layer 16 having a composition of 50% CoCrMo and 50% Ti6Al4V.

According to possible embodiments, described using for example FIGS. 2-6, the above single-piece prosthetic component consists exclusively of the substrate 12, of the coating layer 14 and of the one or more intermediate layers 16.

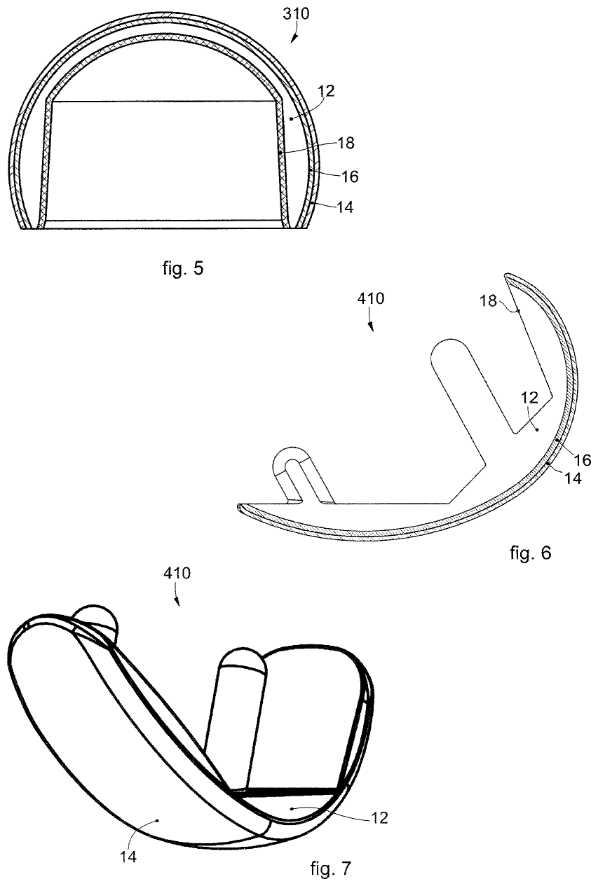

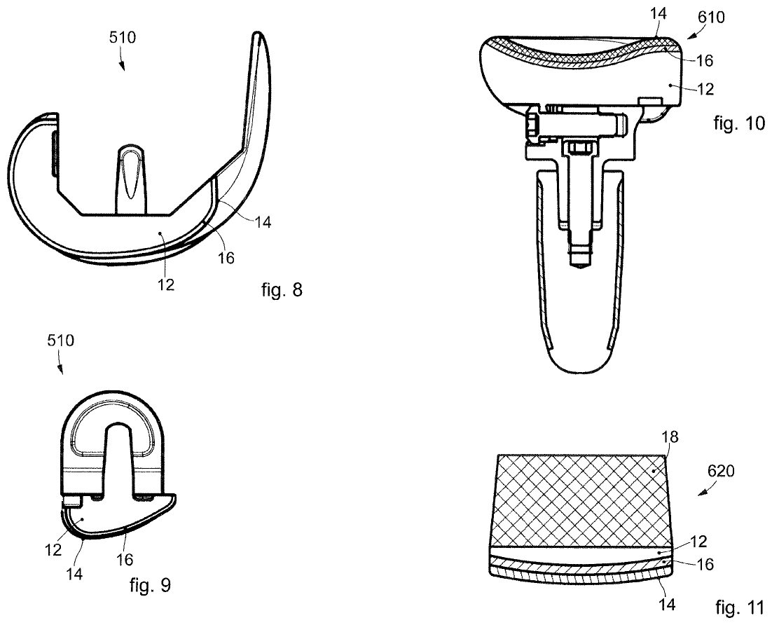

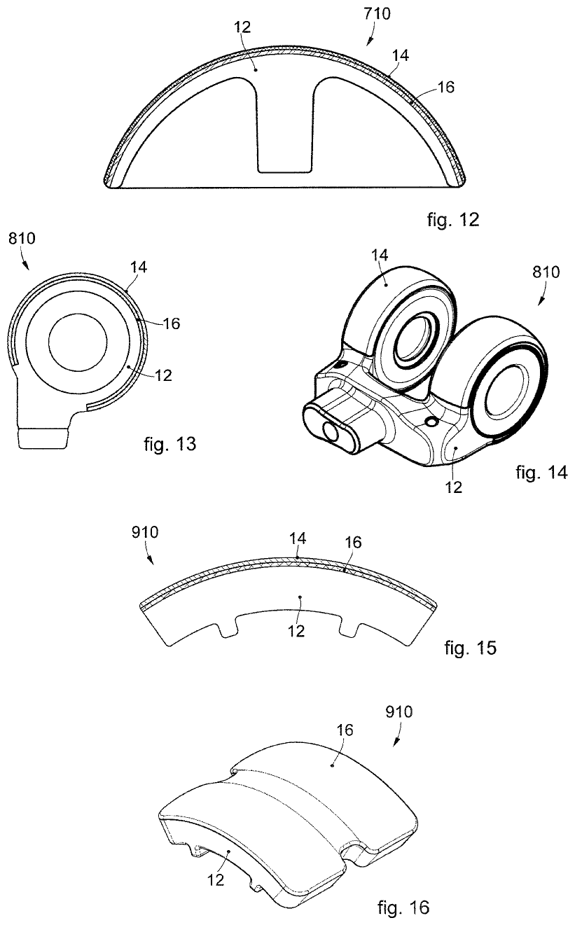

According to possible embodiments, combinable with all the embodiments described here, the above single-piece prosthetic component can be chosen from: a single-piece acetabular cup 110 (see for example FIGS. 1, 2, 3, 19 and 20), a modular acetabular coating, or liner 210 (see for example FIG. 4), a covering or semi-covering prosthesis 310 for a femoral head (see for example FIG. 5), a prosthesis of a femoral component for the knee 410 (see for example FIGS. 6 and 7), a knee prosthesis 510 (see for example FIGS. 8 and 9), a radial capitulum prosthesis 610 (see for example FIG. 10), a prosthesis for focal defects of the cartilage 620 (see for example FIG. 11), a shoulder prosthesis 710 (see for example FIG. 12), an elbow prosthesis 810 (see for example FIGS. 13 and 14), or talar components of an ankle prosthesis 910 (see for example FIGS. 15 and 16).

In particular, by way of a non-limiting example, FIGS. 1, 2 and 3 are used to describe embodiments of the prosthetic component according to the present description which, in the example, is a single-piece acetabular cup 110, for example of the type used for the prosthesis of the femoral head. In this case, the coating layer 14 is present in the internal cavity of the single-piece acetabular cup 110, in order to articulate with the femoral head.

According to possible embodiments, combinable with all the embodiments described here, the substrate 12 can be homogeneous, that is, have a solid and compact structure as shown by way of example in FIGS. 1 and 2.

According to other possible embodiments, combinable with all the embodiments described here, the substrate 12 can have at least partially a porous or trabecular structure. In particular, as described for example in relation to FIG. 3, and applicable to all the other embodiments described here, the substrate 12 can be provided with at least one portion 18 having a porous or trabecular structure, able to promote osseointegration, in particular to improve the adhesion of the prosthetic component to the bone tissue.

According to possible embodiments, the portion 18 could be produced with a porous structure, or in trabecular titanium (TT) by means of additive manufacturing techniques and/or produced in compact form, for example by means of conventional processes, in particular sintering or conventional metallurgical techniques of a subtractive type, such as molding, forging or suchlike.

Further alternative embodiments of the prosthetic component are described using FIGS. 4 to 8. In particular, according to these embodiments, the shapes and functionalities of the prosthetic component can be different according to the type of application required.

For example, FIG. 4 is used to describe, by way of example, embodiments of a prosthetic component according to the present description, which, in the example, is a modular acetabular coating (liner), for example in the form of a cap, indicated with the reference number 210, in which the substrate 12 is opposite the concavity, so as to be inserted, during use, in the bone tissue, while the coating layer 14 is present in a concavity suitable to accommodate, during use, the femoral head or the artificial prosthetic head made of a suitable material, according to the type of prosthesis.

FIG. 5 is used to describe, by way of example, embodiments of a prosthetic component according to the present description, which, in the example, is a coating or semi-coating prosthesis, indicated with the reference number 310, for a femoral head (resurfacing or hemi-resurfacing), which can be associated with the head of a femur. In this case, the substrate 12 faces toward the inside of the concavity, so as to come into contact with the femoral head. For this purpose, the substrate 12 can be provided with a trabecular portion 18 to improve the attachment of the substrate 12 to the bone tissue.

The coating layer 14 is instead disposed on the external surface of the prosthetic component so as to come into contact, during use, with the acetabulum of the pelvis, or the artificial acetabular cup, made of suitable material, according to the type of prosthesis.

FIGS. 6 and 7 are used to describe, by way of example, embodiments of a prosthetic component according to the present description which, in the example, is a prosthesis of a femoral component of the knee, indicated with the reference number 410, in which the substrate 12 faces toward the inside of the concavity so as to come into contact with the patella of a patient.

Advantageously, the substrate 12 of the prosthetic component can be provided with a trabecular portion 18 on its internal surface, so as to improve the attachment to the bone tissue.

Also in this case, the coating layer 14 is disposed on the external surface of the prosthetic component so as to come into contact, during use, with the tibia of the knee, or the liner of the artificial tibial prosthetic component, made of suitable material, according to the type of prosthesis.

FIGS. 8 and 9 are used to describe, by way of example, embodiments of a prosthetic component according to the present description which, in the example, is a prosthesis of a component of the knee, indicated with the reference number 510.

FIG. 10 is used to describe, by way of example, embodiments of a prosthetic component according to the present description which, in the example, is a component of a prosthesis for the radial capitulum, indicated with the reference number 610.

FIG. 11 is used to describe, by way of example, embodiments of a prosthetic component according to the present description which, in the example, is a component of a prosthesis for focal defects of the cartilage, indicated with the reference number 620.

FIG. 12 is used to describe, by way of example, embodiments of a prosthetic component according to the present description which, in the example, is a component of a shoulder prosthesis, indicated with the reference number 710.

FIGS. 13 and 14 are used to describe, by way of example, embodiments of a prosthetic component according to the present description which, in the example, is a component of an elbow prosthesis, indicated with the reference number 810, in particular the humeral condyle.

FIGS. 15 and 16 are used to describe, by way of example, embodiments of prosthetic components according to the present description which, in the example, are talar components of an ankle prosthesis, indicated with the reference number 910.

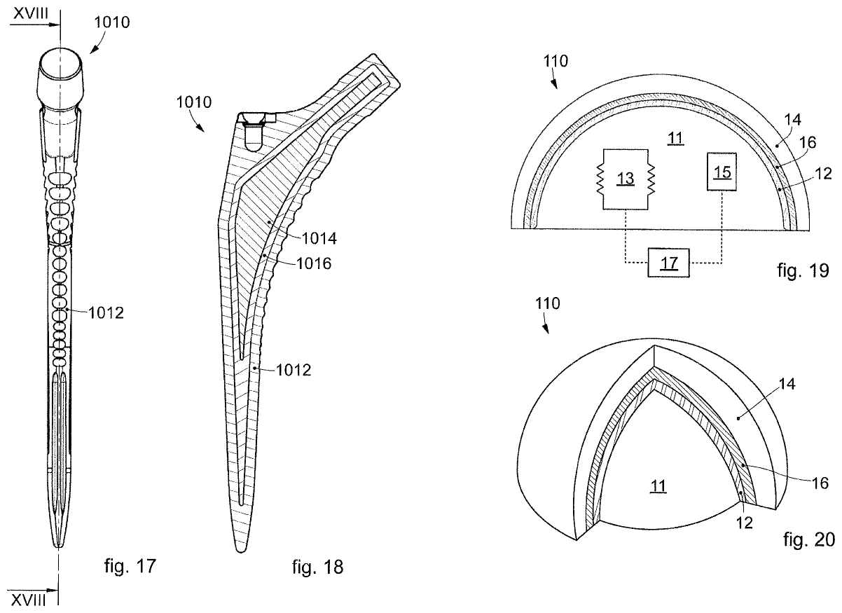

In further embodiments, it is also provided that the method is used to produce a single-piece prosthetic component, in which the cobalt-based alloy constitutes an internal core thereof, while the titanium-based alloy completely covers the internal core defining an external layer, and a surface, suitable for osseointegration, advantageously with a porous or trabecular structure. The internal core made of cobalt-based alloy confers on the prosthetic component thus obtained high mechanical features. For example, these embodiments can be used to obtain a femoral stem prosthesis 1010 (see for example FIGS. 17 and 18), in which there is an internal substrate, or core, 1014, made of cobalt-based alloy, completely coated with a coating layer 1012 made of titanium-based alloy.

In particular, FIGS. 17 and 18 are used to describe, by way of example, embodiments of a prosthetic component according to the present description which, in the example, is a femoral stem prosthesis, indicated with the reference number 1010, in which an internal substrate, or core, 1014 is present which constitutes the core of the structure to exploit its mechanical properties of resistance. This substrate 1014 is made available using conventional production techniques, or by means of a DED process, or laser cladding.

According to possible embodiments, combinable with all the embodiments described here, the production of the coating layer 1012 can provide to make the substrate 1014 by means of a DED process, or laser cladding, advantageously with a porous or trabecular structure, for the purposes of osseointegration, as previously described with reference to the embodiments which provide the substrate 12 made of a titanium-based alloy.

In these embodiments it is also possible to provide one or more intermediate layers 1016 with a functional gradient, as already described above, for example in relation to figs. from 1 to 16, obtained by means of a DED process, or laser cladding, with the same advantages already described above.

FIGS. 19 and 20 are used to describe embodiments, combinable with all the embodiments described here, in which the production method according to the present description provides to use a support 11 on which, for example, the substrate 12, or the substrate, or core, 1014 is made. For descriptive purposes only, the following describes the case in which the support 11 is used to produce the embodiments which provide the substrate 12 made of a titanium-based alloy, the coating layer 14 made of a cobalt-based alloy and the possible one or more intermediate layers 16. Hereafter, in particular, these embodiments are described with reference to the prosthetic component 110, however they can also be applied to embodiments of the prosthetic component obtainable according to the method described here and indicated, in the present description, with the reference numbers 210, 310, 410, 510, 610, 620, 710, 810, 910 or also others. It is also possible to apply these embodiments to the production of a prosthetic component 1010 which provides an internal substrate, or core, 1014 made of a cobalt-based alloy, a coating layer 1012 made of a titanium-based alloy and possibly one or more intermediate layers 1016, as better explained below, for example with reference to FIGS. 17 and 18.

Therefore, according to the embodiments in which the support 11 is used to make the substrate 12 of a titanium-based alloy, the support 11 can be configured with a shape and/or geometry complementary to and mating with the substrate 12 itself.

According to the embodiments described using FIGS. 9 and 10, the substrate 12 can be attached on the above support 11.

According to possible embodiments, the support 11 can have different geometries to allow the correct housing of the substrate 12 as the geometry of the latter varies.

In fact, according to possible embodiments, the substrate 12 can have any geometry whatsoever, according to the shape and the intended use of the prosthetic component.

According to possible embodiments, the support 11 can be configured to control the cooling speed of the substrate 12 and possibly to heat it.

According to possible embodiments, the support 11 can be provided with resistance heaters 13, to heat and control the cooling of the substrate 12 and/or of the support 11.

According to possible embodiments, the support 11 can be provided with thermocouples 15 to measure the temperatures of the support 11, of the substrate 12 and/or of the heaters 13.

According to possible embodiments, the support 11 comprises a control and command unit, or system controller, 17, connected to the heaters 13 and/or to the thermocouples 15 and configured to control and regulate the temperature of the support 11, of the substrate 12 and/or of the heaters 13.

According to possible embodiments, the temperatures detected by the thermocouples 15 can be monitored in real time and/or recorded.

According to possible embodiments, the control and command unit 17 can be configured to determine whether the necessary temperature has been reached by means of the thermocouples 15 and operate to maintain it constant.

According to possible embodiments, the heating can occur by means of the laser or electron beam, that is by using the laser or electron beam itself of the DED process without feeding the powder flow to be deposited.

Alternatively or additionally, the resistance heaters 13 of the support 11 can be configured to maintain the support 11 at the temperature necessary to make the coating layer 14 on the substrate 12.

According to possible embodiments, the method according to the embodiments described here can also comprise a step of making, in physical and structural continuity between the substrate 12 and the coating layer 14, at least one, or more than one, intermediate layer 16 made of a material having a functional gradient, with a mixed composition of a cobalt-based and titanium-based alloy by means of a DED process, or laser cladding.

The one or more intermediate layers 16 are each formed, therefore, by a material having a functional gradient, in which the chemical composition of the material varies gradually between the substrate 12 and the coating layer 14.

The one or more intermediate layers 16 thus achievable allow, advantageously, to improve the interface between the substrate 12 and the coating layer 14, avoiding the formation of cracks, and thus giving stability and functionality to the prosthetic component.

According to possible implementations, the composition of the at least one intermediate layer 16 is Ti6Al4V—CoCrMo, according to the formula CoCrMo X%+Ti6Al4V (100−X)% in which X<100.

According to possible embodiments, the method can provide to subject the substrate 12 and the possible intermediate layers 16 to a cooling and to a subsequent intermediate heating. After applying the substrate 12 and/or the last intermediate layer 16, the coating layer 14 is then made.

Subsequently, the prosthetic component is cooled in a controlled manner until it reaches ambient temperature, typically 25° C. at 1 atm.

The coating layer 14 can be brought to the desired thickness, to the desired surface finishing condition and made to comply with the design tolerances by means of conventional processes.

According to possible embodiments, the thickness of the coating layer 14 and of the individual intermediate layers 16 can be regulated as a function of the geometry of the substrate 12 and the final application.

According to possible embodiments, the thickness of each intermediate layer 16 and of the coating layer 14 can be independent of the thickness of the other intermediate layers 16 and of the coating layer 14.

By way of a non-limiting example, in possible embodiments, four intermediate layers 16 are interposed between the substrate 12 and the coating layer 14. In other possible embodiments, two, three, five or even more than five intermediate layers 16 can be present.

According to possible embodiments, the intermediate layers 16 can have the same thickness to one another. For example, the thickness can be comprised between 0.1 mm and 0.3 mm.

In other embodiments, the intermediate layers 16 can have different thicknesses from one another.

According to some embodiments, the method to produce a prosthetic component 110 provides, for example if four intermediate layers 16 are present, the following steps:
making a first intermediate layer 16 on the substrate 12, the first intermediate layer 16 having a composition of 12.5% CoCrMo and 87.5% Ti6Al4V;
making a second intermediate layer 16 on the first intermediate layer 16, the second intermediate layer 16 having a composition of 25% CoCrMo and 75% Ti6Al4V;
making a third intermediate layer 16 on the second intermediate layer 16, the third intermediate layer 16 having a composition of 37.5% CoCrMo and 62.5% Ti6Al4V;
making a fourth intermediate layer 16 on the third intermediate layer 16, the fourth intermediate layer 16 having a composition of 50% CoCrMo and 50% Ti6Al4V.

According to these embodiments, the coating layer 14 is made by means of a Directed Energy Deposition, DED, process, or laser cladding, after the one or more intermediate layers 16 specifically provided have been made. In the case where for example four intermediate layers are provided, the coating layer 14 can be made after the fourth intermediate layer 16 has been made.

According to possible embodiments, the overall thickness of the four intermediate layers 16 and of the coating layer 14 can reach up to 1.2 mm.

However, it is not excluded that the overall thickness of the intermediate layers 16 and of the coating layer 14 can be reduced following a mechanical finishing operation on a machine. For example, the prosthetic component can be subjected to a polishing operation. Furthermore, it is not excluded that the prosthetic component can be subjected to mechanical operations of turning or milling for suitable prosthetic geometries.

According to some embodiments, combinable with all the embodiments described above, the coating layer 14 and/or the possible intermediate layers 16 can be made with an initial thickness that is greater than the final thickness desired, so as to avoid possible reductions following a mechanical finishing or machining operation.

The embodiments described using FIGS. 19 and 20 can be applied, suitably modified where necessary, also to produce prosthetic components according to the embodiments described above using FIGS. 17 and 18. In this case, in the above description, reference will be made respectively to the substrate, or core, 1014, instead of to the substrate 12, to the coating layer 1012 instead of to the coating layer 14, and to the one or more intermediate layers 1016, instead of to the one or more intermediate layers 16.

It is clear that modifications and/or additions of parts and/or steps may be made to the method to produce a prosthetic component 110, 210, 310, 410, 510, 610, 620, 710, 810, 910, 1010 and to the prosthetic component 110, 210, 310, 410, 510, 610, 620, 710, 810, 910, 1010 thus produced as described heretofore, without departing from the field and scope of the present invention.

It is also clear that, although the present invention has been described with reference to some specific examples, a person of skill in the art shall certainly be able to achieve many other equivalent forms of method to produce a prosthetic component and prosthetic component thus produced, having the characteristics as set forth in the claims and hence all coming within the field of protection defined thereby.

In the following claims, the sole purpose of the references in brackets is to facilitate reading and they must not be considered as restrictive factors with regard to the field of protection claimed in the specific claims.

1. Method to produce a single-piece prosthetic component (110, 210, 310, 410, 510, 610, 620, 710, 810, 910), said method comprising:
making available a substrate (12) made of a first titanium-based metal alloy;
making, on said substrate (12), a coating layer (14) of a second cobalt-based metal alloy, in particular cobalt-chromium, by means of a Directed Energy Deposition (DED) process, or laser cladding.

2. Method as in claim 1, wherein said substrate (12) is able to act as a bone interface, being made of a material configured to promote osseointegration.

3. Method as in claim 1, wherein said step of making available said substrate (12) provides to make said substrate (12) by means of a process of additive manufacturing and/or by conventional techniques, in particular sintering or metallurgical techniques of a subtractive type, such as molding, forging or suchlike.

4. Method as in claim 1, wherein said coating layer (14) is able to act as an articular structure, being made of a material configured to resist cyclic loads of wear and friction.

5. Method as in claim 1, wherein said first titanium-based metal alloy is Ti6Al4V.

6. Method as in claim 1, wherein said second cobalt-based metal alloy, in particular cobalt-chromium, is CoCrMo.

7. Method as in claim 1, wherein said method provides to make, in physical and structural continuity between said substrate (12) and said coating layer (14), one or more intermediate layers (16) made with a material having a functional gradient, with a mixed composition of a cobalt-based and titanium-based alloy, using a Directed Energy Deposition process (DED), or laser cladding.

8. Method as in claim 7, wherein said one or more intermediate layers (16) each have a Ti6Al4V—CoCrMo composition, according to the formula
CoCrMo X%+Ti6Al4V(100−X)%
wherein X<100.

9. Method as in claim 7, wherein said step of making one or more intermediate layers (16) provides to:
make a first intermediate layer (16) on said substrate (12), said first intermediate layer (16) having a composition of 12.5% CoCrMo and 87.5% Ti6Al4V;
make a second intermediate layer (16) on said first intermediate layer (16), said second intermediate layer (16) having a composition of 25% CoCrMo and 75% Ti6Al4V;
make a third intermediate layer (16) on said second intermediate layer (16), said third intermediate layer (16) having a composition of 37.5% CoCrMo and 62.5% Ti6Al4V;
make a fourth intermediate layer (16) on said third intermediate layer (16), said fourth intermediate layer (16) having a composition of 50% CoCrMo and 50% Ti6Al4V.

10. Single-piece prosthetic component comprising a substrate (12) made of a first titanium-based metal alloy and, on said substrate (12), a coating layer (14) of a second cobalt-based metal alloy, in particular cobalt-chrome, said coating layer (14) being obtained by a Directed Energy Deposition (DED) process, or laser cladding.

11. Single-piece prosthetic component as in claim 10, wherein said substrate (12) is able to act as a bone interface, being made of a material configured to promote osseointegration.

12. Single-piece prosthetic component as in claim 11, wherein said substrate (12) is obtained by a process of additive manufacturing and/or by conventional techniques, in particular sintering or metallurgical techniques of a subtractive type, such as molding, forging or similar.

13. Single-piece prosthetic component as in claim 10, wherein said coating layer (14) is able to act as an articular structure, being made of a material configured to resist cyclic loads of wear and friction.

14. Single-piece prosthetic component as in claim 10, wherein said first titanium-based metal alloy is Ti6Al4V.

15. Single-piece prosthetic component as in claim 10, wherein said second cobalt-based metal alloy, in particular cobalt-chromium, is CoCrMo.

16. Single-piece prosthetic component as in claim 10, wherein said single-piece prosthetic component (110, 210, 310, 410, 510, 610, 620, 710, 810, 910) comprises, in physical and structural continuity between said substrate (12) and said coating layer (14), one or more intermediate layers (16) made with a material having a functional gradient, with a mixed composition of a cobalt-based and titanium-based alloy, using a Directed Energy Deposition (DED) process, or laser cladding.

17. Single-piece prosthetic component as in claim 16, wherein said one or more intermediate layers (16) each have a composition of Ti6Al4V—CoCrMo, according to the formula
CoCrMo X%+Ti6Al4V(100−X)%
wherein X<100.

18. Single-piece prosthetic component as in claim 16, wherein said one or more intermediate layers (16) comprise:
a first intermediate layer (16) on said substrate (12), said first intermediate layer (16) having a composition of 12.5% CoCrMo and 87.5% Ti6Al4V:
a second intermediate layer (16) on said first intermediate layer (16), said second intermediate layer (16) having a composition of 25% CoCrMo and 75% Ti6Al4V;
a third intermediate layer (16) on said second intermediate layer (16), said third intermediate layer (16) having a composition of 37.5% CoCrMo and 62.5% Ti6Al4V;
a fourth intermediate layer (16) on said third intermediate layer (16), said fourth intermediate layer (16) having a composition of 50% CoCrMo and 50% Ti6Al4V.

19. Single-piece prosthetic component as in claim 10, wherein said single-piece prosthetic component consists exclusively of said substrate (12) and of said coating layer (14).

20. Single-piece prosthetic component as in claim 16, wherein said single-piece prosthetic component consists exclusively of said substrate (12), of said coating layer (14) and of said one or more intermediate layers (16).

21. Single-piece prosthetic component as in claim 10, wherein said single-piece prosthetic component is selected from: a single-piece acetabular cup (110), a modular acetabular coating, or liner (210), a coating or semi-coating prosthesis (310) for a femoral head, a prosthesis of a femoral component for the knee (410), a knee prosthesis (510), a radial capitulum prosthesis (610), a prosthesis for focal defects of the cartilage (620), a shoulder prosthesis (710), an elbow prosthesis (810), talar components of an ankle prosthesis (910).

22. Method to make a single-piece prosthetic component (1010), said method comprising:
making available a substrate, or core, (1014) made of a first cobalt-based metal alloy;
making, on said substrate (1014), a coating layer (1012) of a second titanium-based metal alloy, by means of a Directed Energy Deposition (DED) process, or laser cladding.

23. Single-piece prosthetic component comprising an internal substrate, or core, (1014) made of a first cobalt-based metal alloy and, on said substrate (1014), a coating layer (1012) of a second titanium-based metal alloy, which completely covers said internal substrate, or core, (1014), said coating layer (1012) being obtained by a Directed Energy Deposition (DED) process, or laser cladding.

24. Single-piece prosthetic component as in claim 23, wherein said single-piece prosthetic component is a femoral stem prosthesis (1010).
